(19) United States
(12) Patent Application Publication
(10) Pub. No.: US 2006/0085016 A1
(43) Pub. Date: Apr. 20, 2006

(54) Suture instrument and method of suturing in cosmetic surgery

(76) Inventor: Sorin Eremia, Riverside, CA (US)

Correspondence Address: EZRA SUTTON, PLAZA 9, 900 ROUTE 9, WOODBRIDGE, NJ, 07095, US

(21) Appl. No.: 10/966,197

(22) Filed: Oct. 15, 2004

(51) Int. Cl. A61B 17/04

(52) U.S. Cl. 606/148

(57) ABSTRACT

A method of treating a patient in cosmetic surgery, using first and second hollow tubular surgical members and a suture to be surgically inserted under the patient's facial skin, including the following steps: cutting a single incision in the patient's facial skin adjacent to the area of the ear; threading the first and second hollow tubular members with a continuous suture having thereon a plurality of spaced-apart, movable cross sutures; inserting the insertion tip with the threaded suture through the incision and under the facial skin, toward the patient's fold line in the nasal area; removing the second hollow tubular member from the first hollow tubular member and from the nasal area and out through the single incision, while leaving in place the suture and the plurality of cross sutures so they are disposed in the facial tissue; pulling on the proximal tip of the suture which is extending out of the incision in order to pull the plurality of cross sutures in the facial tissue which causes the cross sutures to move from their substantially parallel relationship to the suture so that they are moved to a position which is substantially perpendicular to the suture in order to engage and anchor the cross sutures in the facial tissue; pulling and tieing off the suture in the area of the incision which operates to pull the facial tissue and the facial fold line more tightly in order to minimize the appearance of the facial fold line in the nasal area.

### FIELD OF THE INVENTION

The present invention relates to a novel suture instrument and a novel method of suturing using the novel suture instrument in cosmetic, plastic, or reconstructive plastic surgery. More particularly, the invention relates to a two component suture having a main suture and a plurality of cross sutures thereon, wherein a two member suture instrument is used to perform a single entry incision (point) for placing the two component suture therein.

### BACKGROUND OF THE INVENTION

Cosmetic, facial, plastic and reconstructive surgeons have constantly sought new and improved-surgical devices, sutures and methods of surgery to aid in the support of physical anatomical structures that have lost their natural tension and support. The most often surgically treated areas include the face, the breast/chest region, the buttocks, the thighs and other anatomical areas that lose tension and sag. Current surgical devices and methods are not adequate in providing optimum natural-looking physical appearance to prevent such loss of tension in these structures.

There remains a need for a suturing system and method having a plurality of spaced-apart, cross sutures thereon that support body tissue being augmented by facial surgery, breast surgery, or buttock surgery, using a novel two member surgical (cosmetic) instrument on the patient.

### DESCRIPTION OF THE PRIOR ART

Suspension sutures, anchor sutures, suture implements, and suturing instruments having various designs, configurations, structures and materials of construction have been disclosed in the prior art. For example, U.S. Pat. No. 5,480,405 to YOON discloses an anchor applier instrument for use in suturing tissue. The suturing instrument extends through a portal in the anatomical cavity wall for manipulating a needle to penetrate the tissue to form a looped portion of a length of suture material at an exit point in the tissue and to back the needle out of the tissue, and the anchor applier instrument extends through another portal in the anatomical cavity wall for positioning an anchor in engagement with the looped portion of the suture material to allow the suture material to be tensioned and secured to adjustably approximate the tissue. This prior art patent does not disclose or teach the structure and configuration of the two member suture instrument of the present invention, or its method of use.

U.S. Pat. No. 6,599,310 to LEUNG et al. discloses a method for joining and holding closed a wound in bodily tissue using a barbed suture including sharp pointed ends. This prior art patent does not disclose or teach the structure and configuration of the two member suture instrument of the present invention, or its method of use.

U.S. Publication No. 2002/0029011 to DYER discloses devices that are directed to laminated materials which are used to provide support under tension for body structures. These laminated devices can be used in methods for facial, plastic, cosmetic and reconstructive surgery. This prior art patent does not disclose or teach the structure and configuration of the two member suture instrument of the present invention, or its method of use.

None of the aforementioned prior art patents teach or disclose a two component suspension suture having a main suture and a plurality of spaced-apart, movable cross sutures thereon for use with a two member suture instrument.

Accordingly, it is an object of the present invention to provide a two member suture instrument that allows for a single entry incision (point) in order to place a two component suture in the facial tissue, breast tissue or buttock tissue, for use in cosmetic, plastic, facial and reconstructive plastic surgery.

Another object of the present invention is to provide a suture instrument having a first hollow tubular member and a second hollow tubular member for receiving the two component suture therethrough.

Another object of the present invention is to provide a two component suspension suture having a main suture and a plurality of spaced-apart, movable cross sutures thereon that support the tissue being augmented by the surgery using the two member suture instrument.

Another object of the present invention is to provide a two component suspension suture wherein the multiple cross sutures move to a substantially perpendicular position having a wider cross suture after tension is applied to the suspension suture which engages the facial tissue at multiple points as the tissue is being pulled to tighten it.

Another object of the present invention is to provide a two component suspension suture wherein the main suture and cross suture system resolves existing problems of dimpling, back slippage of the pulled facial tissue, and/or strangulating and cutting off the blood supply of the pulled tissue, or trapping of a nerve within the pulled tissue when using classic suturing techniques (the suture is passed through the tissue and then doubled back to pull the tissue up like a pulley).

Another object of the present invention is to provide a two member suture instrument in conjunction with a two component suspension suture having a plurality of cross sutures that can be mass produced in an automated and economical manner and is readily affordable by patients, surgeons and hospitals.

### SUMMARY OF THE INVENTION

In accordance with the present invention, there is provided a method of treating a patient in cosmetic surgery, using first and second hollow tubular surgical members and a suture to be surgically inserted under the patient's facial skin, including the following steps: cutting a single incision in the patient's facial skin adjacent to the area of the ear; threading the first and second hollow tubular members with a continuous main suture having thereon a plurality of spaced-apart, movable cross sutures; inserting the insertion tip with the threaded suture through the incision and under the facial skin, toward the patient's fold line in the nasal area; removing the second hollow tubular member from the first hollow tubular member and from the nasal area and out through the single incision, while leaving in place the main suture and the plurality of cross sutures so they are disposed in the facial tissue; pulling on the proximal tip of the suture which is extending out of the incision in order to pull the plurality of cross sutures in the facial tissue which causes the cross sutures to move from their substantially parallel relationship to the suture to a position which is substantially perpendicular to the suture in order to engage and anchor the cross sutures in the facial tissue; pulling and tieing off the suture in the area of the incision which operates to pull the facial tissue and the facial fold line more tightly in order to minimize the appearance of the facial fold line in the nasal area.

### BRIEF DESCRIPTION OF DRAWINGS

Further objects, features and advantages of the present invention will become apparent upon the consideration of the following detailed description of the presently preferred embodiment when taken in conjunction with the accompanying drawings, wherein:

FIG. 1 is a perspective view of the two member suture instrument and the two component suspension suture system of the preferred embodiment of the present invention showing the major component parts in an assembled state being in operational use thereof;

FIG. 2 is a perspective view of the two member suture instrument and the two component suspension suture system of the present invention showing a first hollow tubular member and a suspension suture having a main suture and a plurality of cross sutures for anchoring or attaching within the facial tissue;

FIG. 3 is a perspective view of the two member suture instrument and the two component suspension suture system of the present invention showing the first hollow tubular member and the multiple cross sutures or anchoring sutures being anchored or attached within the facial tissue adjacent to the facial fold line;

FIG. 4 is a perspective view of the two component suspension suture system of the present invention showing the pulled facial tissue from the attached and pulled multiple cross sutures of the suspension suture in its pulled up position;

FIG. 5 is a perspective view of the two component suspension suture system of the present invention showing the pulled up fold line within the facial tissue using three suspension suture systems;

FIG. 6 is a perspective view of the two member suture instrument and the two component suspensions suture system of the present invention showing a first hollow tubular member, a second hollow tubular member and a suspension suture having thereon a plurality of spaced-apart, movable cross sutures for threading within the first and second hollow tubular members;

FIG. 7A is an enlarged perspective view of the two member suture instrument of the present invention showing distal ends of the first and second hollow tubular members being interconnected with each other;

FIG. 7B is an enlarged perspective view of the two member suture instrument of the present invention showing distal ends of the first and second hollow tubular members being interconnected with each other; and

FIG. 8 is a detailed perspective view of the two component suspension suture system of the present invention showing the main suture having a plurality of cross sutures thereon.

### DETAILED DESCRIPTION OF THE PREFERRED EMBODIMENT

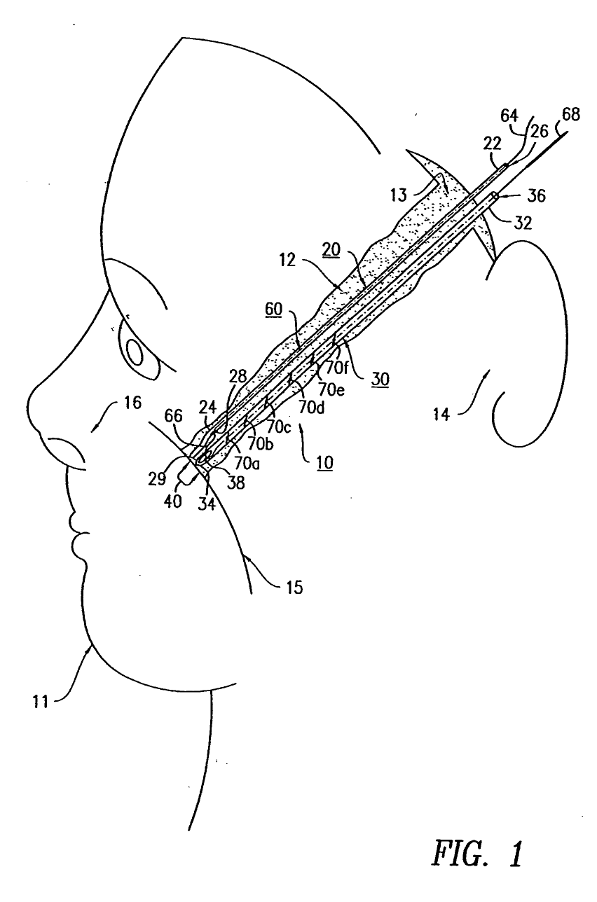

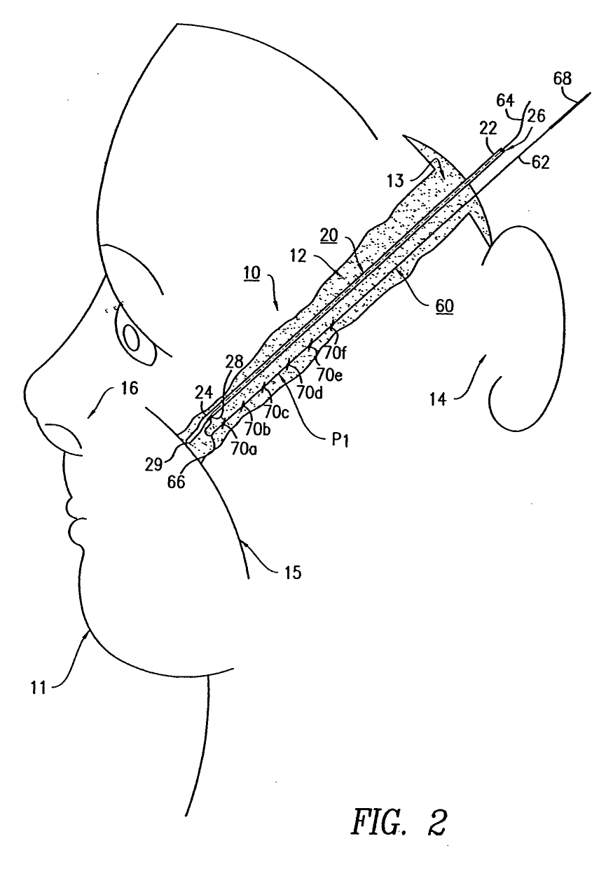

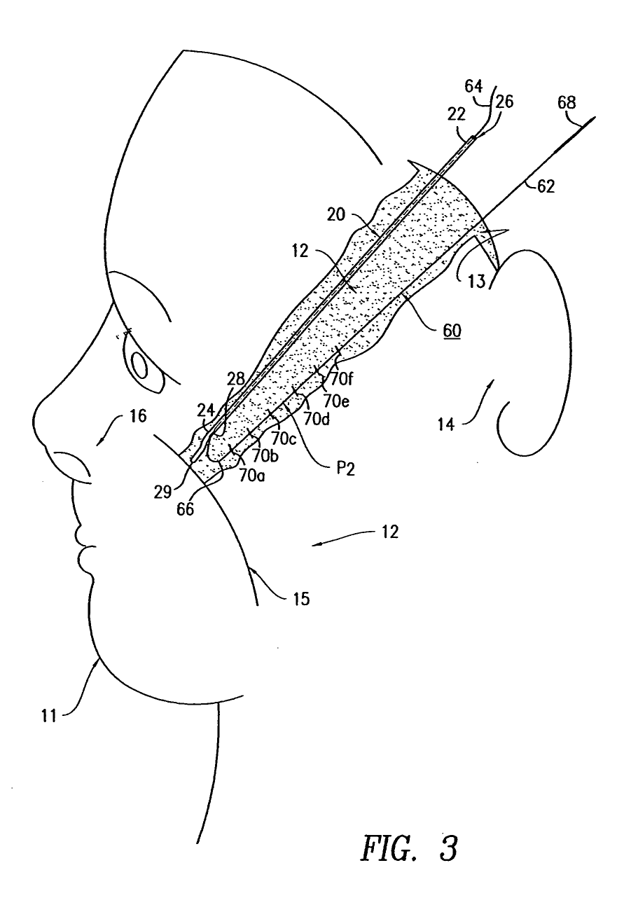

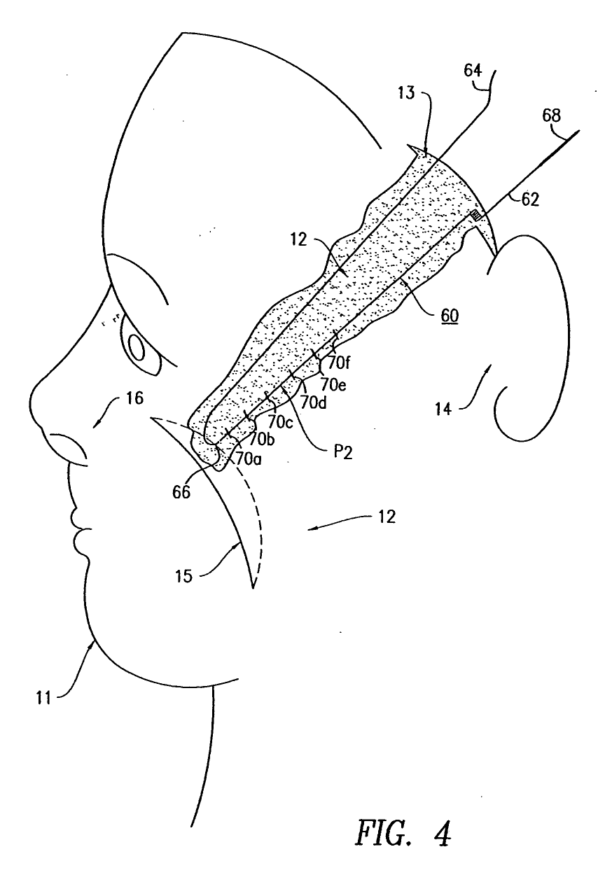

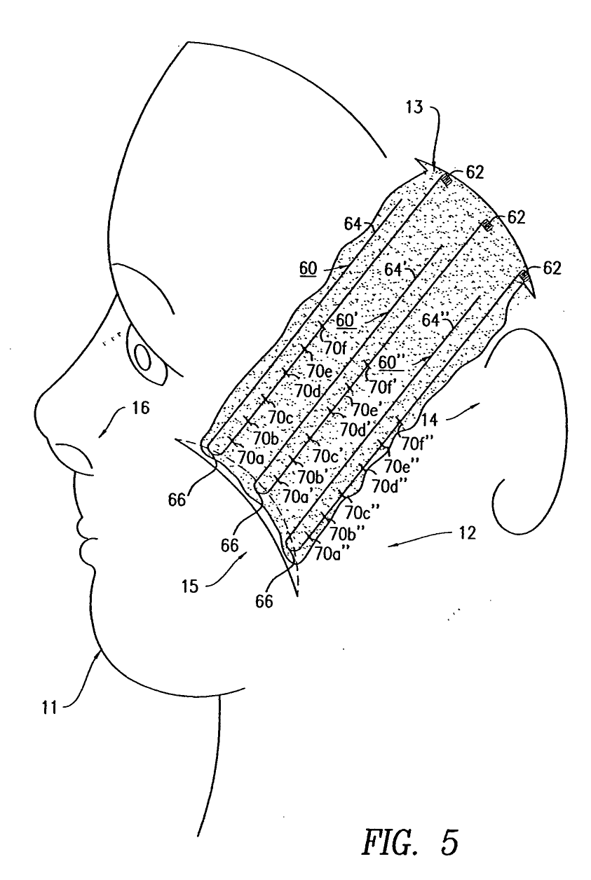

The two member surgical instrument 10 and its component parts of the preferred embodiment of the present invention are represented in detail by FIGS. 1 through 8 of the patent drawings. The two member surgical instrument 10 and the two component suspension suture system 60 are used in cosmetic, plastic, facial and reconstructive plastic surgery for the augmentation of facial tissue 12 or other tissue, as shown in FIGS. 1 through 5.

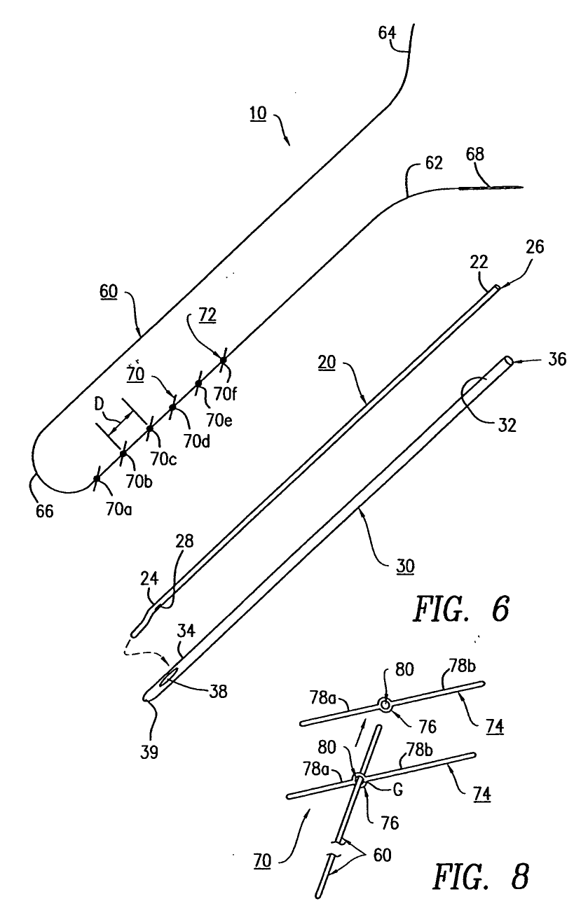

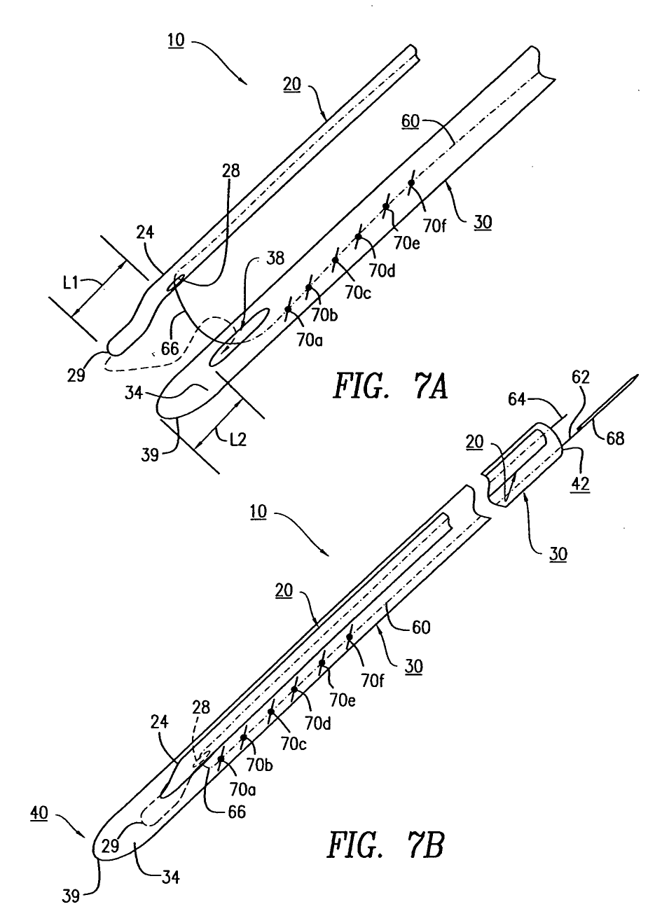

As shown in FIGS. 1, 6 and 7, the surgical instrument 10 of the present invention includes a first hollow tubular member 20 having a proximal end 22 and a distal end 24, with a first suture opening 26 therethrough, as well as a second hollow tubular member 30 having a proximal end 32 and a distal end 34, with a second suture opening 36 therethrough. The distal end 24 of the first hollow tubular member 20 also includes a suture outlet opening 28 for allowing the suspension suture 60 to pass through outlet opening 28. The distal end 34 of the second hollow tubular member 30 further includes a suture opening 38 and a conical tip 39 being adjacent to suture opening 38. The suture opening 38 is used for receiving insert tip 29 of distal end 24 of the first hollow tubular member 20 for forming a joint insertion tip 40, as shown in FIG. 7 of the drawings, for inserting into an incision 13.

The two component suspension suture system 60 includes a first end 62, a second end 64, and a mid-section 66. First end 62 includes a needle tip 68 fixedly attached to the first end 62, as shown in FIG. 1. The main suture 60 also includes along the mid-section 66 a plurality of spaced-apart, movable cross sutures or suture anchors 70a, 70b, 70c, 70d, 70e and 70f being disposed towards the first end 62, as depicted in FIG. 1 of the drawings. The main suture 60 is threaded through respective suture openings 26, 28, 38 and 36 of first and second hollow tubular members 20 and 30, respectively, such that the needle tip 68 is inserted in the proximal end 22 of tubular member 20 where then the needle tip 68 passes out of the suture opening 28 at the distal end 24 of tubular member 20. The needle tip 68 is then inserted into the opening 38 at the distal end 34 of tubular member 30 and then the needle tip 68 passes out of the opening 36 of tubular member 30. The foregoing steps are referred to herein as the threading operation.

A “cross suture” or “suture anchor” is defined as a short piece of cross suture 70a or 70b connected to the main suture 60 by:

a) knots 72 on the main suture 60 being spaced apart a distance D of 10 mm±5 mm;

b) welding a short piece of cross suture 70 to the main suture 60;

c) a short piece of cross suture 70 having [—o—] the configuration 74 of a suture ring 76 in the center of two opposite suture pieces 78a and 78b, with the suture ring 76 having an opening 80 for receiving the main suture 60 therethrough (See FIG. 8); and

d) cross sutures or suture anchors 70a to 70f are connected to the main suture 60 in any of the following ways: by knots, or laser welding, or gluing G, or epoxy gluing.

The first hollow tubular member 20 has a length of 12 cms±2 cms, an 18 gauge diameter (1 mm±1 mil), and a wall thickness of 2 mils±0.5 mil. The second suture opening 26 has an oval shape with a width of 1 mm±1 mil and a length of 2 mm±1 mil. The second suture opening 26 is positioned at a distance L1 of 5.5 mm±1 mil from the distal end 24 of the first hollow tubular member 20.

The second hollow tubular member 30 has a length of 12 cms±2 cm, an 14 gauge diameter (2 mm±1 mil) and a wall thickness of 2 mils±0.5 mil. The fourth suture opening 36 has an oval shape with a width of 1.5 mm±1 mil and a length of 6 mm±1 mil. The fourth suture opening 36 is positioned at a distance L2 of 3 mm±1 mil from the distal end 34 of the second hollow tubular member 30.

The first and second hollow tubular members 20 and 30 are made from polished stainless steel (re-sterilizeable) or from disposable (throw away) and sterilizeable hard plastics.

The main suture 60 has a length of 40 cms±5 cms and a diameter of 0.2 mm±1 mil. The spaced-apart, movable cross sutures 70a to 70f have a spaced distance D of 10 mm±5 mm between the cross sutures 70a to 70f. The main suture 60 is a clear 2-0 monofilament, slow dissolving suture (comparable to a PDS) having a straight (¾ inch to 1 inch) needle 68 attached thereto, or it can be a permanent suture.

### METHOD OF THE PRESENT INVENTION

The new method of treating a patient 11 using cosmetic surgery is shown in FIGS. 1 to 5 of the drawings. The method uses the new instrument 10 of the present invention and the new suture 60. Using the new method, the first and second hollow tubular members 20 and 30 and the suture 60 are surgically inserted under the patient's facial skin 12, and the new method includes the following steps:

a) cutting a short single incision 13 (about ½ inches in length) in the patient's facial skin 12 adjacent to the area of the ear 14;

b) threading the first and second hollow tubular members 20 and 30 with the continuous main suture 60 having thereon a plurality of spaced-apart, movable cross sutures or suture anchors 70a to 70f so that the cross sutures 70a to 70f are disposed in the second hollow tubular member 30 with the cross sutures 70a to 70f substantially parallel to the axis of the second tubular member 30;

c) placing the distal 24 end of the first hollow tubular member 20 into an opening 38 formed near the distal end 34 of the second hollow tubular member 30 so that the first and second hollow tubular members 20 and 30 are substantially parallel to each other and in contact with each other and thereby form an insertion tip 40 and a proximal tip 42;

d) inserting the insertion tip 40 with the threaded suture 60 through the incision 13 and under the facial skin 12, toward the patient's fold line 15 in the nasal area 16;

e) removing the second hollow tubular member 30 from the first hollow tubular member 20 and from the nasal area 16 and out through the single incision 13, while leaving in place the suture 60 and the plurality of cross sutures 70a to 70f so they are disposed in the facial tissue and being substantially parallel to the first hollow tubular member 20 which is still in place;

f) pulling on the proximal tip 62 of main suture 60 which is extending out of the incision 13 in order to pull the plurality of cross sutures 70a to 70f in the facial tissue 12 which causes the cross sutures 70a to 70f to move from their substantially parallel relationship to the main suture 60 to a position which is substantially perpendicular to the main suture 60 in order to engage and anchor the cross sutures 70a to 70f in the facial tissue 12; the cross sutures 70a to 70f are movable from a non-anchoring first position P₁ to an anchoring second position P2 within the facial tissue 12 of a patient 11;

g) removing the first hollow tubular member 20 from the nasal area 16 and out through the single incision 13 while leaving in place in the facial tissue 12 the main suture 60 and the plurality of anchored cross sutures 70a to 70f;

h) pulling and tieing off the proximal tip of the main suture 60 in the area of the incision 13 which operates to pull the facial tissue 12 and the facial fold line 15 more tightly in order to minimize the appearance of the facial fold line 15 in the nasal area 16; and

i) repeating steps b) through h) recited above one or more times to place one or more additional main sutures 60′ and 60″ and their respective cross sutures 70a′ to 70f′ and 70a″ to 70f″ in the facial tissue 12 in a substantially parallel relationship to the main suture 60 which operates to further pull the facial tissue 12 and the facial fold line 15 more tightly in order to minimize the appearance of the facial fold line 15 in the nasal area 16.

### ADVANTAGES OF THE PRESENT INVENTION

Accordingly, an advantage of the present invention is that it provides for a two member suture instrument that allows for a single entry incision (point) in order to place a two component suture in the facial tissue, breast tissue or buttock tissue, for use in cosmetic, plastic, facial and reconstructive plastic surgery.

Another advantage of the present invention is that it provides for a suture instrument having a first hollow tubular member and a second hollow tubular member for receiving the two component suture therethrough.

Another advantage of the present invention is that it provides for a two component suture having a main suture and a plurality of spaced-apart, movable cross sutures thereon that support the tissue being augmented by the surgery using the two member suture instrument.

Another advantage of the present invention is that it provides for a two component suspension suture wherein the multiple cross sutures move to a substantially perpendicular position having a wider cross suture after tension is applied which engages the facial tissue at multiple points as the tissue is being pulled to tighten it.

Another advantage of the present invention is that it provides for a two component suspension suture wherein the main suture and cross suture system resolves existing problems of dimpling, back slippage of the pulled facial tissue, and/or strangulating and cutting off the blood supply of the pulled tissue, or trapping of a nerve within the pulled tissue when using classic suturing techniques (the suture is passed through the tissue and then doubled back to pull the tissue up like a pulley).

Another advantage of the present invention is that it provides for a two member suture instrument in conjunction with a two component suspension suture having a plurality of cross sutures that can be mass produced in an automated and economical manner and is readily affordable by patients, surgeons and hospitals.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed is:

1. A method of treating a patient using cosmetic surgery, using first and second hollow tubular members and a suture to be surgically inserted under the patient's facial skin, including the following steps:
a) cutting a single incision in the patient's facial skin adjacent to the area of the ear;
b) threading the first and second hollow tubular members with a continuous first suture having thereon a plurality of spaced-apart, movable cross sutures so that said cross sutures are disposed in said second hollow tubular member with the cross sutures substantially parallel to the axis of said second tubular member;
c) placing the distal end of the first hollow tubular member into an opening formed near the distal end of the second hollow tubular member so that the first and second hollow tubular members are substantially parallel to each other and in contact with each other and thereby form an insertion tip and a proximal tip;
d) inserting the insertion tip with the threaded first suture through the incision and under the facial skin, toward the patient's fold line in the nasal area;
e) removing the second hollow tubular member from the first hollow tubular member and from the nasal area and out through the single incision, while leaving in place the suture and the plurality of cross sutures so they are disposed in the facial tissue and being substantially parallel to the first hollow tubular member which is still in place;
f) pulling on the proximal tip of the suture which is extending out of the incision in order to pull the plurality of cross sutures in the facial tissue which causes the cross sutures to move from their substantially parallel relationship to the suture so that the cross sutures under traction are moved to a position which is substantially perpendicular to the suture in order to engage and anchor the cross sutures in the facial tissue;
g) removing the first hollow tubular member from the nasal area and out through the single incision while leaving in place in the facial tissue the suture and the plurality of anchored cross sutures;
h) pulling and tieing off the first suture in the area of the incision at the proximal tip of the suture which operates to pull the facial tissue and the facial fold line more tightly in order to minimize the appearance of the facial fold line in the nasal area; and
i) repeating steps b) through h) recited above one or more times to place one or more additional sutures and their respective cross sutures in the facial tissue in a substantially parallel relationship to the first suture which operates to further pull the facial tissue and the facial fold line more tightly in order to minimize the appearance of the facial fold line in the nasal area.

2. A method of treating a patient using cosmetic surgery in accordance with claim 1, wherein the step of pulling includes moving said plurality of cross sutures from a non-anchoring position to an anchoring position within the facial tissue of a patient.

3. A method of treating a patient using cosmetic surgery, using first and second hollow tubular members and a suture to be surgically inserted under the patient's skin, including the following steps:
a) cutting a single incision in the patient's skin;
b) threading the first and second hollow tubular members with a continuous first suture having thereon a plurality of spaced-apart, movable cross sutures so that said cross sutures are disposed in said second hollow tubular member with the cross sutures substantially parallel to the axis of said second tubular member;
c) placing the distal end of the first hollow tubular member into an opening formed near the distal end of the second hollow tubular member so that the first and second hollow tubular members are substantially parallel to each other and in contact with each other and thereby form an insertion tip and a proximal tip;
d) inserting the insertion tip with the threaded first suture through the incision and under the skin;
e) removing the second hollow tubular member from the first hollow tubular member and out through the single incision, while leaving in place the first suture and the plurality of cross sutures so they are disposed in the tissue and being substantially parallel to the first hollow tubular member which is still in place;
f) pulling on the proximal tip of the suture which is extending out of the incision in order to pull the plurality of cross sutures in the tissue which causes the cross sutures to move from their substantially parallel relationship to the first suture so that the cross sutures are moved to a position which is substantially perpendicular to the first suture in order to engage and anchor the cross sutures in the tissue;
g) removing the first hollow tubular member and out through the single incision while leaving in place in the tissue the first suture and the plurality of anchored cross sutures;
h) pulling and tieing off the first suture in the area of the incision at the proximal tip of the first suture which operates to pull the tissue more tightly in order to minimize the appearance of the fold line; and
i) repeating steps b) through h) recited above one or more times to place one or more additional sutures and their respective cross sutures in the tissue in a substantially parallel relationship to the first suture which operates to further pull the tissue more tightly in order to minimize the appearance of the fold line.

4. A method of treating a patient using cosmetic surgery in accordance with claim 3, wherein the step of pulling includes moving said plurality of cross sutures from a non-anchoring position to an anchoring position within the tissue of a patient.

5. A suture instrument in combination with a suspension suture for use in cosmetic surgery, comprising:
a) a first hollow tubular member having a first proximal end and a first distal end;
b) said first proximal end of said first hollow tubular member having a first suture opening, and said first distal end of said first hollow tubular member having an insert tip and a second suture opening adjacent to said insert tip;
c) said first hollow tubular member for receiving a suspension suture therethrough;
d) said insert tip at said first distal end of said first hollow tubular member being offset from the longitudinal axis of said first hollow tubular member;
e) a second hollow tubular member having a second proximal end and a second distal end;
f) said second proximal end of said second hollow tubular member having a third suture opening, and said second distal end of said second hollow tubular member having a conical tip and a fourth suture opening adjacent to said conical tip;
g) said second hollow tubular member for receiving said suspension suture therethrough;
h) said fourth suture opening of said second distal end of said second hollow tubular member for receiving said insert tip of said first distal end of said first hollow tubular member such that said first and second hollow tubular members are substantially parallel with each other and in contact with each other in order to form an insertion tip for inserting into an incision in the tissue of a patient;
i) said suspension suture including a first suture end, a mid-section and a second suture end;
j) said mid-section of said suspension suture having thereon a plurality of spaced-apart, movable cross sutures; and
k) said plurality of spaced-apart, movable cross sutures being movable from a non-anchoring first position to an anchoring second position within the tissue of a patient.

6. A suture instrument in combination with a suspension suture in accordance with claim 5, wherein said first hollow tubular member has a length of 12 cms±2 cms, a diameter of 1.0 mm±1 mil and a wall thickness of 2.0 mils±0.5 mil.

7. A suture instrument in combination with a suspension suture-in accordance with claim 5, wherein said second suture opening of said first hollow tubular member has an oval shaped opening.

8. A suture instrument in combination with a suspension suture in accordance with claim 5, wherein said second suture opening has a width of 1.0 mm±1 mil and a length of 2.0 mm±1 mil.

9. A suture instrument in combination with a suspension suture in accordance with claim 5, wherein said second suture opening is positioned at a distance of 5.5 mm±1 mil from said distal end of said first hollow tubular member.

10. A suture instrument in combination with a suspension suture in accordance with claim 5, wherein said second hollow tubular member has a length of 12 cms±2 cms, a diameter of 2.0 mm±1 mil, and a wall thickness of 2.0 mils±0.5 mil.

11. A suture instrument in combination with a suspension suture in accordance with claim 5, wherein said fourth suture opening of said second hollow tubular member has an oval shaped opening.

12. A suture instrument in combination with a suspension suture in accordance with claim 5, wherein said fourth suture opening has a width of 1.5 mm±1 mil and a length of 6.0 mm±1 mil.

13. A suture instrument in combination with a suspension suture in accordance with claim 5, wherein said fourth suture opening is positioned at a diameter of 3.0 mm±1 mil from said distal end of said second hollow tubular member.

14. A suture instrument in combination with a suspension suture in accordance with claim 5, wherein said first and second hollow tubular members are made from sterilizeable polished stainless steel.

15. A suture instrument in combination with a suspension suture in accordance with claim 5, wherein said first and second hollow tubular members are made from sterilizeable disposable hard plastics.

16. A suture instrument in combination with a suspension suture in accordance with claim 5, wherein said distal end of said first hollow tubular member has a conically-shaped tip.

17. A suture instrument in combination with a suspension suture in accordance with claim 5, wherein said distal end of said second hollow tubular member has a conically-shaped tip.

18. A suture instrument in combination with a suspension suture in accordance with claim 5, wherein said suspension suture has a length of 40 cms±5 cms and a diameter 0.2 mm 1 mil.

19. A suture instrument in combination with a suspension suture in accordance with claim 5, wherein said movable sutures have a spaced distance between said movable cross sutures of 10 mm±5 mm.

20. A suture instrument in combination with a suspension suture in accordance with claim 5, wherein said first suture end of said suspension suture includes a detachably connected needle.
